(19) United States
(12) Patent Application Publication
(10) Pub. No.: US 2003/0097115 A1
(43) Pub. Date: May 22, 2003

(54) Closed sterile system devices and methods

(75) Inventor: Micheal Gruenberg, Poway, CA (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: MedCell Biologics, Inc., San Diego, CA

(21) Appl. No.: 09/992,737

(22) Filed: Nov. 19, 2001

(51) Int. Cl. A61M 5/00; A61M 31/00

(52) U.S. Cl. 604/500; 604/171; 604/226

(57) ABSTRACT

The present invention includes a syringe device that includes an elongated tubular barrel having a first end and a second end, a plunger telescopically received within at least a portion of the barrel, such that a portion of the plunger extends from the first end of the barrel, and a protective sheath positioned adjacent to an external surface of the portion of the plunger, such that the protective sheath encloses the portion of the plunger extending outwardly from the first end of the barrel. The present invention further includes methods of transporting a biological material within the syringe device, and transferring the biological material between a source container and the syringe device.

### CROSS-REFERENCE TO RELATED APPLICATION(S)

None

### BACKGROUND OF THE INVENTION

The present invention generally relates to a syringe device. More specifically, the present invention relates to a syringe device that includes a protective sheath to prevent exposure of a barrel to an outside environment during operation of the syringe device. The present invention further includes a method of using the syringe device in a closed sterile biological system.

Mammalian cells are increasingly used in diagnostic and medical applications. For example, mammalian cells maybe used for production of proteins for vaccines, therapeutics and diagnostics. In addition, mammalian cells may be used for adoptive cell therapy or tissue engineering. Furthermore, mammalian cells may be used as part of a medical device that functions as an artificial organ. As a result, there is an increase in demand for mammalian cells and/or mammalian cell-based products. The increase in demand has fueled a need for techniques and/or equipment that promote efficient mammalian cell growth and/or productivity.

Bioreactors have commonly been employed for the cultivation of living organisms as part of a cell culture system. A bioreactor typically includes a housing that contains cells and nutrients maintained at bioreactor conditions that permit cell growth and/or production of secreted products. Critical to any cell culture system is the maintenance of absolute sterility. Since addition to and/or removal of materials from cell culture systems provides an ideal source of microbial contamination, special precautions must be observed during addition and/or withdrawal to the cell culture system. In the past, one approach used for maintaining a sterile environment during cell culture sampling is to carry out the sampling procedure in a biological laminar flow hood. However, this approach is only practical for relatively small cell culture vessels, and quite cost-prohibitive for large scale cell culture vessels.

Another approach for addition to and/or withdrawal from cell culture systems involves the use of a sterile hypodermic needle insertion through a pierceable, self-reasealable, elastomeric septum in an appropriately placed sampling port of a cell culture vessel. However, this approach permits piercing to occur only once, and is generally not adaptable to repeated addition and/or withdrawal in cell culture systems. This has necessitated biological production facilities to operate under clean room conditions of Class 10,000 or greater. Biological manufacturing operations in clean rooms is expensive and labor intensive, especially when operating under a FDA-mandated Good Manufacturing Practice (GMP) conditions.

Medical syringes do not provide a closed, sterile environment for use in biological production. These syringes are sterilized with the plunger fully inserted into the syringe barrel. When these syringes are removed from their protective coverings, the barrel is exposed to the outside environment and is thus no longer sterile. As the plunger is pulled up the barrel, the material collected is exposed to the non-sterile barrel environment. Thus current syringe designs do not provide a completely closed, sterile environment for the repeated withdrawal and addition of materials. Therefore, there exists an urgent need to develop a cost-effective, efficient method to add and/or withdraw samples from a cell culture system in a sterile manner in a non-clean room environment.

### BRIEF SUMMARY OF THE INVENTION

The present invention includes a syringe device that includes an elongated tubular barrel having a first end and a second end, a plunger telescopically received within at least a portion of the barrel, such that a portion of the plunger extends from the first end of the barrel, and a protective sheath positioned adjacent to an external surface of the portion of the plunger, such that the protective sheath encloses the portion of the plunger extending outwardly from the first end of the barrel. The present invention further includes methods of transporting a biological material within the syringe device, and transferring the biological material between a source container and the syringe device. The present invention permits repeated addition and/or withdrawal to cell culture systems in a sterile manner in a non-clean room environment.

### BRIEF DESCRIPTION OF THE DRAWINGS

FIG. 1 is a longitudinal sectional view of the present invention showing an extended protective sheath.

FIG. 2 is a longitudinal sectional view of the present invention showing a compressed protective sheath.

FIG. 3 is a longitudinal sectional view of the present invention showing an alternate extended protective sheath.

FIG. 4 is a longitudinal sectional view of the present invention showing an alternate compressed protective sheath.

FIG. 5 is a sectional view of an alternate embodiment that includes two devices of the present invention.

### DETAILED DESCRIPTION

The present invention generally relates to a syringe device. More specifically, the present invention relates to a syringe device that includes a protective sheath to prevent exposure of a barrel to an outside environment during operation of the syringe device. The present invention further includes a method of using the syringe device in a closed sterile biological system. The syringe device is also beneficial for use with patents that are at risk of infection due to weakened immune systems.

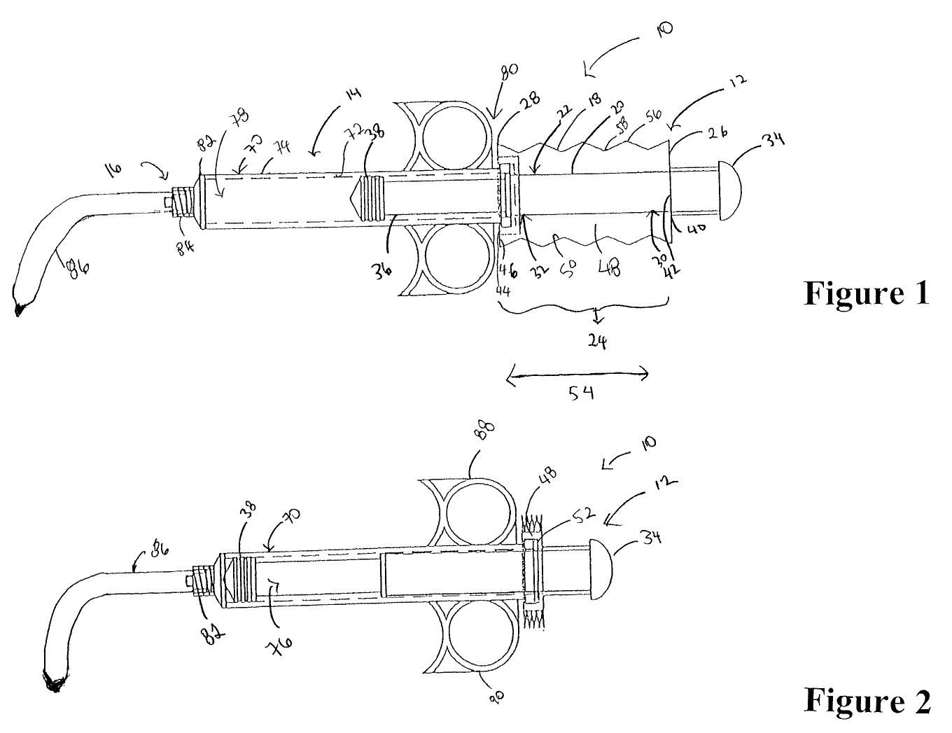

A syringe device that includes a protective sheath is generally depicted at 10 in FIG. 1. The syringe device 10, hereinafter referred to as the “device 10” includes a protected portion 12, a cylindrical mid-portion 14 and a discharge portion 16. Like reference characters will be used to indicate like elements throughout FIGS. 1-4.

The protected portion 12 further includes a protective sheath 18 that completely encloses and/or surrounds a plunger 20 of the device 10. The protective sheath 18 is typically positioned adjacent to an external surface 22 of the plunger 20.

Preferably, the protective sheath 18 completely surrounds a portion 24 of the plunger 20 extending from the mid-portion 14 of the device 10 that would be exposed to an outside environment during operation of the device 10. Still more preferably, the protective sheath 18 surrounds the portion 24 of the plunger 20 that extends from the mid-portion 14 of the device 10 during retraction and/or compression of the plunger 20 such that the portion 24 is never exposed to the outside environment.

The protective sheath 18 has a first end 26 and a second end 28. Similarly, the plunger 20 has a first end 30 and a second end 32. The first end 30 of the plunger 20 terminates with a handle portion 34. The plunger 20 further includes a piston 36 attached to a seal 38 that is enclosed within the mid-portion 14 of the device 10. The piston 36 and seal 38 slidably engage the mid-portion 14 during operation of the device 10.

The first end 26 of the protective sheath 18 is connected to the first end 30 of the plunger 20 through a first annular wall 40 lying in a plane that is perpendicular to a longitudinal axis (not shown) of the plunger 20. The first annular wall 40 defines a first annular surface 42 which may serve as a first stop surface 42 during operation of the plunger 20.

The second end 28 of the protective sheath 18 is connected to the second end 32 of the plunger 20 through a second annular wall 44 lying on a plane that is perpendicular to the longitudinal axis (not shown) of the plunger 20. The second annular wall 44 defines a second annular surface 46 which may serve as a second stop surface 46 for the plunger 20 during operation of the device 10.

The protective sheath 18 defines an annular recess 48 that encloses the plunger 20. Annular recess 48 is further defined by the first annular surface 42 and the second annular surface 46. Annular recess 48 is consistently protected from exposure to the outside environment by the protective sheath 18 when using the device 10 of the present invention. Annular recess 48 may optionally include additional components, such as air, sterilizing and/or sanitizing gases like nitrogen, oxygen, ozone or any other gases that are compatible with, and that do not interfere with, the operation, content, nor any portion of the device 10.

Preferably, annular recess 48 separates the protective sheath 18 from the plunger 20, such that the protective sheath 18 does not connect with, nor contact the portion 24 the plunger 20 when operating the device 10. When the protective sheath 18 is not directly connected to the plunger 20, and the plunger 20 does not come in direct contact with the protective sheath 18, the plunger 20 is maintained in a sterile environment that is required for sterile transfer during cell culture and closed sterile cell culture systems. Though descriptions of the present device 10 are primarily made in terms of a protective sheath 18 that does not directly connect with, nor directly contact the portion 24 of the plunger 20, it is to be understood that the present device 10 may include a protective sheath 18 that does connect with, and contact the portion 24 of the plunger 20, while still realizing benefits of the present invention, so long as the protective sheath 18 and the portion 24 of the plunger 20 are maintained in a sterile manner that facilitates sterile compression and/or retraction during operation of the device 10.

In addition, an outer diameter (not shown) of the plunger 20 and an inner peripheral surface 50 of the protective sheath 18 are sized so that the external surface 22 of the plunger 20 is received within the annular recess 48 in a manner that facilitates longitudinal movement 52 of the plunger 20 in a direction towards or away from the discharge end 16 of the device 10. Furthermore, a plunger stop 54, as best depicted in FIGS. 2 and 4 may be placed adjacent to the second annular surface 46 to halt any further longitudinal movement of the plunger 20 during operation of the device 10.

The plunger stop 54 may be any member that is effective to halt longitudinal movement during retraction or operation of the plunger 20. As an example, the plunger stop 54 may be in the form of an O-ring, a gasket, or a plastic cylindrical member that is best depicted in FIGS. 2 and 4.

The plunger stop 54 may be generally attached to the mid-portion 14 of the device 10 via a locking mechanism (not shown) that permits easy and rapid assembly or disassembly of the entire protected portion 12 onto or from the mid-portion 14. Easy and rapid assembly and disassembly of the protected portion 12 eases assembly and disassembly of the device 10 during cleaning of the device 10, for example. In addition, the plunger stop 52 prevents inadvertent removal of the plunger 20 from the device 10 and therefore, prevents undesired exposure of the plunger 20 to the outside environment.

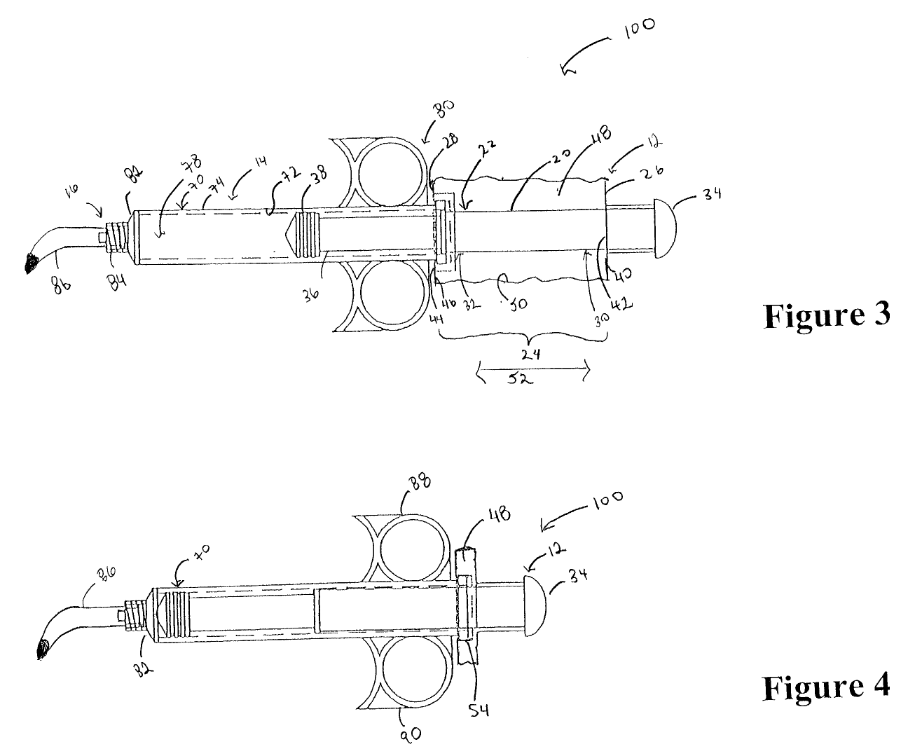

The protective sheath 18 may be secured to the first annular wall 40 and the second annular wall 44 in any conventional manner, such as via an adhesive (not shown), or a circular clamp (not shown) so long as the device 10 may be maintained and/or operated in a sterile manner. The second annular surface 46 is spaced at a predetermined longitudinal distance from the first annular surface 42, such that during operation, the plunger 20 may be moved from initial rearward position as shown by 52 in FIGS. 1 and 3, to a position where the protective sheath 18 abuts the plunger stop 54, to effect a predetermined fluid dosage discharge from the device 10. Therefore, the plunger stop 54 further permits accurate and consistent control of an amount of fluid that may be discharged from the device 10.

Though descriptions of the present invention are primarily made in terms of including the plunger stop 52, it is to be understood that the device 10 does not need to include the plunger stop 54 to realize the benefits of the present invention. First annular wall 40 and second annular wall 44 may also function to halt longitudinal movement 54 of the plunger 20 during operation of the device 10.

Typically, the distance from the first annular surface 42 and the second annular surface 46 may be any distance that permits (1) partial or (2) full longitudinal movement 52 of the piston 36 of the plunger 20 during operation of the device 10. As an example, the distance between the first annular surface 42 and the second annular surface 46 may range from about 2 inches to about 3 inches in length.

The protective sheath 18 may be in the form of an elongated tubular member that has one or more circumferential integral accordion-like pleats that provide one or more alternating circumferential peaks 56 and valleys 58, or an elongated tubular member, as best depicted in FIGS. 1-4. Though protective sheath 18 is shown in FIGS. 1-4 as including an accordion-like or cylindrical tubular design, it will be understood that the protective sheath 18 may have cross-sectional configurations of any form so long as the protective sheath 18 completely encloses and/or surrounds the portion 24 of the plunger 20, when practicing the present invention. In addition, the protective sheath 18 may be, and is preferably, an axially collapsible protective sheath 18 that is capable of being compressed and/or retracted during operation of the device 10.

The protective sheath 18 maybe made of any flexible material, such as glass, plastic or any other suitable flexible material that is effective to axially collapse, enclose, and protect the plunger 20 that extends from the mid-portion 14 during compression and/or retraction of the plunger 20. As indicated, a diameter of the protective sheath 18 that is greater than the outer diameter (not shown) of the plunger 20 is required so that the protective sheath 18 encloses the portion 24 of the plunger 20 that extends from the mid-portion 14 of the device 10.

In general, any conventional material, such as plastic, rubber or stainless steel may be used to form the plunger 20 when practicing the present invention. In addition, the plunger 20, the handle portion 34, piston 36 and the seal 38 may each be formed from the same materials or from different materials.

The mid-portion 14 of the device 10 typically includes a cylindrical tubular barrel 70 that houses the piston 36 and seal 38 of the plunger 20. The barrel 70 may also be characterized as an elongate annular member. Barrel 70 typically has an internal cylindrical surface 72 and an external cylindrical surface 74. The piston 36 and seal 38 of the plunger 20 are received within the barrel 70 to seal and slidally engage the internal surface 72 of the barrel 70.

The barrel 70 may be formed from a transparent, relatively thin-walled material, such as plastic, glass, or the like. Furthermore, the external surface 74 may bear indicia (not shown) along the longitudinal length of the external surface 72 to indicate an amount of fluid (not shown) contained within a chamber 78 defined within the barrel 70 by seal 38 of the plunger 20. The chamber 78 may also be adapted to receive and retain a non-fluid substance, such as beads, or other solid and/or semi-solid substance. In addition, the barrel 70 at an opposite end 80 to the discharge portion 16 of the device 10 may be temporarily opened to allow for telescopic insertion of the piston 36 and seal 38 of the plunger 20 into the chamber 78 during assembly of the device 10.

A discharge end 82 of the barrel 70 may have an integral reduced diameter portion 84 that receives and retains a discharge member 86, such as tubing 86, a needle (not shown), a receiving device (not shown), or any combination thereof. The discharge member 86 may be permanently or temporarily affixed to the device 10, depending on an intended number of uses of the device 10. As an example, the discharge end 82 may be provided with a standard coupling feature, such as a Luer lock coupling or bayonet type coupling for affixing the discharge member 86 the barrel 70.

When the discharge member 86 is tubing, the tubing may have an occluded end such that the tube end is closed to the outside environment to prevent, and preferably eliminate, ingress or egress of fluid, microorganisms, or any other contaminating matter. As an example, the end of the tubing may be sealed via heat, solvent or the like, as best depicted in FIGS. 1-4.

To facilitate manipulation of the device 10 and the plunger 20, a pair of holders 88 and 90 may be provided. Holders 88 and 90 may be formed from the same material as the barrel 70 or from a different material as the barrel 70. The holders 88 and 90 may be formed of any suitable material so long as the holders 88 and 90 permit the operator (not shown) to efficiently compress and/or retract the plunger 20 during operation of the syringe device 10. Holders 88 and 90 may be integrally formed with, adhesively attached to, or otherwise connected to the barrel 70.

As an example, holders 88 and 90 may include a pair of diametrically opposed radially extending grips that can be held by the operator's fingers, as best in depicted in FIGS. 1-4. In this manner, the device 10 may be readily placed in the operator's hand with the holders 88 and 90 between two fingers (not shown) and the operator's thumb (not shown) engaging the handle portion 34 to facilitate handling and manipulation during operation.

Though the device 10 is shown in FIGS. 1-4 as including a generally cylindrical design, it will be understood that the device 10 may have cross sectional configurations of any form within the present invention. In addition, the protective sheath 18 of the present invention may apply to a device 10 of varying configurations.

In use, as the handle portion 34 of the plunger 20 is advanced, fluid within the barrel 70 is forced through the discharge end 82. The protective sheath 18 collapses axially as the portion 24 of the plunger 20 is pushed longitudinally towards the discharge end 82 so that the portion 24 of the plunger 20 is never exposed during discharge. Similarly, when the handle portion 34 is withdrawn, fluid is taken into the barrel 70 through the discharge end 82. Furthermore, the protective sheath 18 extends axially during withdrawal of the plunger 20 so that the portion 24 of the plunger 20 is never exposed during withdrawal. Thus, during operation of the device 10, the plunger 20 is consistently protected from exposure to the outside environment by the protective sheath 18 that compresses or extends in response to the movement of the plunger 20.

The device 10 provides an effective and yet easy tool to add and/or withdraw samples from closed biological systems in a sterile manner, such as adding and/or withdrawing samples from a closed cell culture system since the protective sheath 18 is able to protect the portion 24 of the plunger 20 that is outside of the barrel 70. The protective sheath 18 provides protection of the plunger 20 before, during and after use of the device 10 and shields the plunger 20 against exposure to the outside environment. In addition, the protective sheath 18 is typically inexpensive to make and the attachment thereof to the device 10 is easily accomplished.

The device 10 of the present invention may be used as a transport device (not shown) to transport a biological material from a first location to a second location, or to add and/or withdraw one or more samples in a sterile manner.

When the device 10 is used as a transport device, the discharge end 82 is typically sealed to prevent exposure of the sample to the outside environment. When the device 10 is used to add and/or withdraw one or more samples in a sterile manner, the device 10 is preferably combined with Sterile Tubing Welding (STW) technology to facilitate transfer of one or more samples from the device 10 to one or more receiving members. As used herein, the terms “STW technology” and “sterile tubing welding technology” refer to at least one apparatus and/or at least one system that is effective to connect, join or splice together two or more tubes in a functionally closed sterile system.

All references to “STW technology” and “sterile tubing welding technology” is understood as including at least one cutting apparatus that is effective to cut the sterile and/or closed end tubes, at least one heating apparatus that is effective to heat the cut sterile and/or closed end tubes, at least one pair of mounting blocks adapted to receive, hold and flatten the tubes to be joined, and/or at least one apparatus that is effective to provide movement, alignment, separation, or urging between the blocks, such that the tubes are connected, spliced together and/or joined in a sterile manner.

All references to “STW technology” or “sterile tubing welding technology” is also understood as including at least one method of connecting, splicing or joining two or more sterile and/or closed ends tubes. The method may generally include (1) flattening two tubes that are to be joined in an appropriate section so that inside walls of the tubes meet, (2) melting through the flattened tubes, (3) sealing the melted or molten tube ends, (4) moving into alignment the melted or molten tube ends, (5) pushing the melted or molten tube ends together to form a joint, (6) cooling the joined tube ends, and/or (7) subjecting the joined cooled ends to slight stress to open the temporary seal in each tube to thereby affect fluid communication between the joined tubes that are connected to the device 10 and the receiving member. Additionally, the method preferably occurs within a functionally closed sterile system.

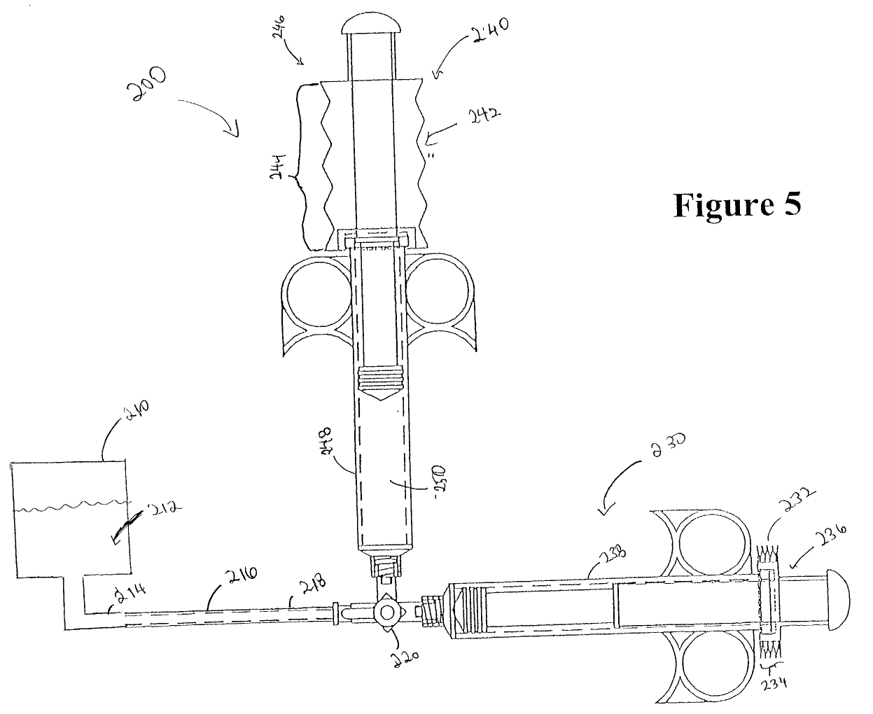

An example of using the device 10 with STW technology is generally depicted in FIG. 5. Initially, a source container 210 containing a biological material 212 that is in fluid communication with a plastic tube 214 is joined to a plastic tube 216 using an STW technology apparatus. The joined plastic tube 216 was derived from plastic tube 214 and plastic tube 218 that is attached to a valve 220. Valve 220 is simultaneously connected to media device 230 and air device 240. The joined plastic tube 216 places the source container 210 in fluid communication with either the media device 230 or the air device 240 through the valve 220.

Next, the valve 220 is turned to permit the media device 230 to withdraw a desired amount of the biological material 212 form the source container 210. As the biological material 212 is withdrawn from the source container 210, a protective sheath 232 surrounds and/or completely encloses a portion 234 of a plunger 236 that is outside of a barrel 238 of the media device 230 to prevent exposure of the portion 234 during retraction of the plunger 236. The protective sheath 232 extends axially during retraction of the plunger 236 to thereby prevent exposure of the portion 234 of the plunger 236 that remains outside of the barrel 238 to an outside environment.

Next, the valve 220 is turned to close off fluid communication between the source container 210 and the media device 230 and opened to permit fluid communication between the source container 210 and the air device 240. The air device includes a protective sheath 242 that completely encloses and/or surrounds a portion 244 of a plunger 246 that extends outside of barrel 248. The barrel 248 is typically filled with air 250 that is injected into the joined plastic tube 216 through the valve 220. As the plunger 246 is depressed, air 250 is discharged from the barrel 248 through valve 220 and into plastic tubing 218. The protective sheath 232 collapses axially during compression of the plunger 246 to thereby prevent exposure of the portion 244 of the plunger 246 that remains outside of the barrel 248 to the outside environment during operation of the air device 240. The air 250 from the air device 240 is used to flush out any biological material 212 that is collected and/or entrapped in the valve 220 or the plastic tube 216.

Flushing out the valve 220 and the joined plastic tube 216 permits removal of any biological material 212 residing in the plastic tube 216 or the valve 220. In addition, flushing out the valve 220 and the joined plastic tube 216 helps minimize any losses of biological material 212 by moving any biological material 212 that may have been entrapped in the valve 220 and the plastic tube 216 back into the source container 210. Furthermore, injecting air 250 into the valve 220 and the plastic tube 216 also helps to maintain sterile withdrawal of biological material product 212 from the source container 210 into the media device 230.

After flushing, the joined plastic tube 216 is cut (not shown) in a sterile manner to thereby disconnect fluid communication between the source container 210, the media device 230 and the air device 240. In addition, the tube ends (not shown) for plastic tube 214 and plastic tube 218 may be heat sealed to eliminate any ingress or egress of any contaminating matter, such as microorganisms, fluid, or the like into the source container 210, the media device 230 or the air device 240. An example of a suitable apparatus and/or system that may be used to join or connect tubes in accordance with the present invention is the Terumo® Sterile Tubing Welder TSCD SC-201A that is available from Terumo Corporation of Tokyo, Japan. An example of a suitable apparatus and/or system that may be used to cut the joined plastic tube 216, and optionally seal the ends of plastic tubes (not shown) individually connected to the source container 210 or the valve 220 is Terumo® Tube Sealer AC-155 that is also available from Terumo Corporation of Tokyo, Japan.

When the device 10 uses STW technology, the discharge member 86 is preferably plastic tubing formed from thermoplastic resin that melts upon application of heat. Some non-exhaustive examples of suitable thermoplastic resins that may used as the plastic tubing in accordance with the present device include polyethylene, polypropylene, polystyrene, poly vinyl chloride, copolymers of polyethylene, copolymers of polypropylene, copolymers of polystyrene, copolymers of poly vinyl chloride, or any combination of any of these.

Though descriptions of the present invention are primarily made in terms of the preferred plastic tubing, it is to be understood that any other tubing, such as glass tubing, rubber tubing, stainless steel tubing, or the like, may be used in accordance with the present invention. Likewise, it is to be understood that the though the present device 10 includes the use of STW technology to help transfer one or more samples, it is to be understood that any other apparatus, system, and/or method that is effective to weld tubes together, such as any device, either automated or manual, that is capable of welding together tubing of any size, may be used in accordance with the present invention while still realizing the benefits of the present invention.

When the device 10 is used to transfer samples in a closed sterile biological system, such as a cell therapy system or a bioreactor system, the device 10 provides a cost-effective technique to transport and transfer biological samples in a sterile manner. The device 10 is also an efficient tool that eliminates the need for expensive clean room manufacturing equipment and/or facilities typically required as part of the closed biological system since the biological material in a source container may be added to or withdrawn from a collection device using the device 10 without exposure to the outside environment.

Furthermore, the device 10 provides a high degree of flexibility during operation of the closed sterile biological system by permitting the operator to rapidly transfer and/or transport biologicals samples, for example, without having to utilize complicated protocols typically implemented for closed sterile biological systems. In addition, using the device 10 along with STW technology helps to minimize any losses, and eliminates most, if not all waste that typically results in closed sterile biological systems since the present invention may include the step of flushing the tubes involved in the transfer of biological material with air to send any biological material trapped in the tubes back into the source container or device 10.

Although the present invention has been described with reference to preferred embodiments, workers skilled in the art will recognize that changes may be made in form and detail without departing from the spirit and scope of the invention.

1. A syringe device for use in transporting a biological material, the syringe device comprising:
an elongated tubular barrel having a chamber for containing the biological material, wherein the tubular barrel has a first end;
a plunger telescopically received within at least a portion of the barrel, wherein a portion of the plunger extends outwardly from the first end of the barrel; and
a protective sheath positioned adjacent to an external surface of the portion of the plunger extending outwardly from the first end of the barrel.

2. The syringe device of claim 1 wherein the protective sheath comprises an accordion-like pleat.

3. The syringe device of claim 1 wherein the protective sheath comprises a first end and a second end, wherein the first end is attached to a first annular wall located on the plunger, and wherein the second end is attached to a second annular wall located on the plunger.

4. The syringe device of claim 3 wherein the protective sheath defines an annular recess that separates the protective sheath from the plunger.

5. The syringe device of claim 1 wherein the protective sheath protects the portion of the plunger extending outwardly from the barrel during any longitudinal displacement of the plunger.

6. A syringe device for use in transferring a biological material, the syringe device comprising:
an elongated tubular barrel having a first end and a second end, the barrel having a generally cylindrical external surface, the barrel defining a chamber therethrough, and the barrel having a discharge member affixed to the second end;
a plunger telescopically received within at least a portion of the barrel, wherein the plunger has a portion extending outwardly from the first end of the barrel; and
a protective sheath having a first end and a second end, the protective sheath affixed to the first end of the barrel through a first annular wall at the first end of the protective sheath, the protective sheath affixed to the plunger through a second annular wall at the second end of the protective sheath, the protective sheath positioned adjacent to the plunger, and the protective sheath surrounding at least a portion of the plunger received within the barrel.

7. The syringe device of claim 6 wherein the protective sheath encloses the portion of the plunger extending outwardly from the barrel during any longitudinal movement of the plunger.

8. The syringe device of claim 6 wherein the protective sheath protects the portion of the plunger extending outwardly from the barrel from exposure to an outside environment.

9. The syringe device of claim 6 wherein the protective sheath comprises at least one accordion-like circumferential pleat formed therein.

10. The syringe device of claim 6 wherein the protective sheath defines an annular recess that separates the protective sheath from portion of the plunger extending outwardly from the barrel.

11. The syringe device of claim 6 wherein the discharge member is a plastic tube.

12. The syringe device of claim 6 wherein the plastic tube is capable of being welded.

13. The syringe device of claim 6 and further including a plunger stop located adjacent to the second annular wall.

14. The syringe device of claim 6 wherein the plunger stop is effective to halt any longitudinal movement of the plunger.

15. A syringe device for use in transporting a biological material, the syringe device comprising:
a protected portion connected to a barrel portion, the protected portion comprising a protective sheath and a plunger, the protective sheath enclosing at least a portion of the plunger, and the protective sheath terminating at an annular wall that is adjacent to the barrel portion; and
wherein the protective sheath is effective to protect a portion of the plunger extending outwardly from the barrel portion.

16. The syringe device of claim 15 wherein the portion of the plunger is telescopically received within the barrel portion.

17. The syringe device of claim 15 wherein the protective sheath axially collapses or extends during compression or retraction of the plunger to protect the portion of the plunger extending outwardly from the barrel portion from exposure to an outside environment.

18. The syringe device of claim 15 wherein the protective sheath comprises an accordion-like pleat.

19. The syringe device of claim 15 and further including a discharge member.

20. The syringe device of claim 19 wherein the discharge member is a plastic tube.

21. The syringe device of claim 20 wherein the plastic tube is formed from polymers of polyethylene, polypropylene, polystyrene, poly vinyl chloride, copolymers of polyethylene, copolymers of polypropylene, copolymers of polystyrene, copolymers of poly vinyl chloride, or any combination of any of these.

22. The syringe device of claim 15 wherein the barrel portion defines a chamber therein for receiving a fluid.

23. The syringe device of claim 22 wherein the fluid is retained in the chamber.

24. The syringe device of claim 23 wherein the fluid is discharged through the discharge member when the plunger is compressed.

25. A method of transporting a biological material, the method comprising:
placing the biological material in a chamber of a syringe device, the syringe device comprising an elongate tubular barrel that defines the chamber;
positioning a plunger in the elongate tubular barrel, a portion of the plunger extending outside the barrel, a protective sheath enclosing the portion of the plunger that extends outside the barrel; and
moving the syringe from a first location to a second location.

26. The method of claim 25 and further including a discharge member attached to an end of the barrel, wherein the discharge member is occluded.

27. A method of transferring a biological material from a source container to a syringe device, the method comprising:
joining a plastic tube attached to a source container to a discharge member attached to a syringe device to thereby effect fluid communication between the source container and the syringe device, the syringe device comprising an elongate tubular barrel, a plunger and a protective sheath, the barrel defining a chamber, the plunger placed in the chamber, the protective sheath enclosing a portion of the plunger extending outside the chamber; and
transferring the biological material from the source container to the chamber.

28. The method of claim 27 wherein the transfer of biological material occurs in a functionally closed sterile system.

29. A method of transferring a biological material from a source container to a syringe device in a functionally closed system, the method comprising:
forming a joined tube between a plastic tube attached to a source container and a discharge member attached to a valve in a functionally closed system, wherein the valve is simultaneously attached to a first syringe device and a second syringe device, wherein the joined tube permits fluid communication between the source container, the first and the second syringe device, and the first syringe device comprising a first elongate tubular barrel, a first plunger and a first protective sheath, the first barrel defining a first chamber, the first plunger placed in the first chamber, and the first protective sheath enclosing a portion of the first plunger extending outside the chamber; and
transferring the biological material into the first chamber.

30. The method of claim 29 wherein the functionally closed system is a sterile tubing welding apparatus.

31. The method of claim 29 wherein the valve is turned to permit fluid communication between the source container and the first syringe device during transfer.

32. The method of claim 29 and further including turning the valve to prevent fluid communication between the source container and the first syringe device after transfer of the biological material to the first syringe device.

33. The method of claim 29 and further including turning the valve to the second syringe device to permit fluid communication between the source container and the second syringe device.

34. The method of claim 33 wherein the second syringe device comprises a second elongate tubular barrel, a second plunger and a second protective sheath, the second barrel defining a second chamber, the second plunger placed in the second chamber, and the second protective sheath enclosing a portion of the second plunger extending outside the chamber.

35. The method of claim 34 and further including injecting air from the second syringe device into the joined tube to flush out the valve, the joined tube or any combination thereof.

36. The method of claim 35 wherein the second protective sheath is effective to protect any length of the plunger from exposure to an outside environment.

37. A device for use in transporting biological material, the device comprising:
a barrel having a chamber for containing the biological material in a sterile environment;
a plunger slidable within the chamber and having a portion extending outwardly therefrom; and
a protective sheath enclosing in a sterile manner the portion of the plunger extending outwardly from the chamber.

38. The device of claim 37 wherein the portion extending outwardly from the chamber varies in length and the sheath accommodates any length of the plunger extending from the chamber.

39. The device of claim 37 wherein the barrel has an end opposite the plunger for providing a sterile ingress and egress from the biological material.
